(19) United States
(12) Patent Application Publication
(10) Pub. No.: US 2013/0131651 A1
(43) Pub. Date: May 23, 2013

(54) FEATURES PROVIDING LINEAR ACTUATION THROUGH ARTICULATION JOINT IN SURGICAL INSTRUMENT

(71) Applicant: ETHICON ENDO-SURGERY, INC., Cincinnati, OH (US)

(72) Inventors: Geoffrey S. Strobl, Williamsburg, OH (US); Charles J. Scheib, Loveland, OH (US); Chad P. Boudreaux, Cincinnati, OH (US); David K. Norvell, Monroe, OH (US)

(73) Assignee: ETHICON ENDO-SURGERY, INC., Cincinnati, OH (US)

(21) Appl. No.: 13/741,650

(22) Filed: Jan. 15, 2013

Related U.S. Application Data
(63) Continuation-in-part of application No. 13/235,683, filed on Sep. 19, 2011.
(60) Provisional application No. 61/386,117, filed on Sep. 24, 2010.

(51) Int. Cl. A61B 17/00

(52) U.S. Cl. CPC A61B 17/00
USPC 606/1

(57) ABSTRACT

An apparatus for operating on tissue includes an end effector having a first jaw and a second jaw. The first jaw is configured to pivot relative to the second jaw from an open position to a closed position. The end effector also includes a blade member configured to translate within the end effector. An articulation portion is positioned between the end effector and a handpiece. The articulation portion is configured to bend to position the handpiece along a first longitudinal axis and the end effector along a second longitudinal axis. The articulation portion comprises a translation feature operable to translate the blade member.

### PRIORITY

This application is a continuation-in-part of U.S. application Ser. No. 13/235,683, filed Sep. 19, 2011, entitled “Articulation Joint Features for Articulating Surgical Device,” which claims priority to U.S. Provisional Application Ser. No. 61/386,117, filed Sep. 24, 2010, entitled “Articulating Surgical Device,” the disclosures of which are incorporated by reference herein.

### BACKGROUND

A variety of surgical instruments include a tissue cutting element and one or more elements that transmit RF energy to tissue (e.g., to coagulate or seal the tissue). An example of such a device is the ENSEAL® Tissue Sealing Device by Ethicon Endo-Surgery, Inc., of Cincinnati, Ohio. Further examples of such devices and related concepts are disclosed in U.S. Pat. No. 6,500,176 entitled “Electrosurgical Systems and Techniques for Sealing Tissue,” issued Dec. 31, 2002, the disclosure of which is incorporated by reference herein; U.S. Pat. No. 7,112,201 entitled “Electrosurgical Instrument and Method of Use,” issued Sep. 26, 2006, the disclosure of which is incorporated by reference herein; U.S. Pat. No. 7,125,409, entitled “Electrosurgical Working End for Controlled Energy Delivery,” issued Oct. 24, 2006, the disclosure of which is incorporated by reference herein; U.S. Pat. No. 7,169,146 entitled “Electrosurgical Probe and Method of Use,” issued Jan. 30, 2007, the disclosure of which is incorporated by reference herein; U.S. Pat. No. 7,186,253, entitled “Electrosurgical Jaw Structure for Controlled Energy Delivery,” issued Mar. 6, 2007, the disclosure of which is incorporated by reference herein; U.S. Pat. No. 7,189,233, entitled “Electrosurgical Instrument,” issued Mar. 13, 2007, the disclosure of which is incorporated by reference herein; U.S. Pat. No. 7,220,951, entitled “Surgical Sealing Surfaces and Methods of Use,” issued May 22, 2007, the disclosure of which is incorporated by reference herein; U.S. Pat. No. 7,309,849, entitled “Polymer Compositions Exhibiting a PTC Property and Methods of Fabrication,” issued Dec. 18, 2007, the disclosure of which is incorporated by reference herein; U.S. Pat. No. 7,311,709, entitled “Electrosurgical Instrument and Method of Use,” issued Dec. 25, 2007, the disclosure of which is incorporated by reference herein; U.S. Pat. No. 7,354,440, entitled “Electrosurgical Instrument and Method of Use,” issued Apr. 8, 2008, the disclosure of which is incorporated by reference herein; U.S. Pat. No. 7,381,209, entitled “Electrosurgical Instrument,” issued Jun. 3, 2008, the disclosure of which is incorporated by reference herein; U.S. Pub. No. 2011/0087218, entitled “Surgical Instrument Comprising First and Second Drive Systems Actuatable by a Common Trigger Mechanism,” published Apr. 14, 2011, the disclosure of which is incorporated by reference herein; and U.S. Pub. No. 2012/0210223, entitled “Motor Driven Electrosurgical Device with Mechanical and Electrical Feedback,” published Aug. 16, 2012, the disclosure of which is incorporated by reference herein.

In addition, a variety of surgical instruments include a shaft having an articulating section, providing enhanced positioning capabilities for an end effector that is located distal to the articulating section of the shaft. Examples of such devices include various models of the ENDOPATH® endocutters by Ethicon Endo-Surgery, Inc., of Cincinnati, Ohio. Further examples of such devices and related concepts are disclosed in U.S. Pat. No. 7,380,696, entitled “Articulating Surgical Stapling Instrument Incorporating a Two-Piece E-Beam Firing Mechanism,” issued Jun. 3, 2008, the disclosure of which is incorporated by reference herein; U.S. Pat. No. 7,404,508, entitled “Surgical Stapling and Cutting Device,” issued Jul. 29, 2008, the disclosure of which is incorporated by reference herein; U.S. Pat. No. 7,455,208, entitled “Surgical Instrument with Articulating Shaft with Rigid Firing Bar Supports,” issued Nov. 25, 2008, the disclosure of which is incorporated by reference herein; U.S. Pat. No. 7,506,790, entitled “Surgical Instrument Incorporating an Electrically Actuated Articulation Mechanism,” issued Mar. 24, 2009, the disclosure of which is incorporated by reference herein; U.S. Pat. No. 7,549,564, entitled “Surgical Stapling Instrument with an Articulating End Effector,” issued Jun. 23, 2009, the disclosure of which is incorporated by reference herein; U.S. Pat. No. 7,559,450, entitled “Surgical Instrument Incorporating a Fluid Transfer Controlled Articulation Mechanism,” issued Jul. 14, 2009, the disclosure of which is incorporated by reference herein; U.S. Pat. No. 7,654,431, entitled “Surgical Instrument with Guided Laterally Moving Articulation Member,” issued Feb. 2, 2010, the disclosure of which is incorporated by reference herein; U.S. Pat. No. 7,780,054, entitled “Surgical Instrument with Laterally Moved Shaft Actuator Coupled to Pivoting Articulation Joint,” issued Aug. 24, 2010, the disclosure of which is incorporated by reference herein; U.S. Pat. No. 7,784,662, entitled “Surgical Instrument with Articulating Shaft with Single Pivot Closure and Double Pivot Frame Ground,” issued Aug. 31, 2010, the disclosure of which is incorporated by reference herein; and U.S. Pat. No. 7,798,386, entitled “Surgical Instrument Articulation Joint Cover,” issued Sep. 21, 2010, the disclosure of which is incorporated by reference herein.

While several medical devices have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a side elevational view of an exemplary electrosurgical medical device;

FIG. 2 depicts a perspective view of the end effector of the device of FIG. 1, in an open configuration;

FIG. 3 depicts another perspective view of the end effector of the device of FIG. 1, in an open configuration;

FIG. 4 depicts a cross-sectional end view of the end effector of FIG. 2, in a closed configuration and with the blade in a distal position;

FIG. 5 depicts a cross-sectional view of an exemplary articulation section for the shaft of the device of FIG. 1;

FIG. 6A depicts a partial perspective view of an exemplary alternative end effector for incorporation into the device of FIG. 1, with a cutting member positioned at a proximal location;

FIG. 6B depicts a partial perspective view of the end effector of FIG. 6A, with the cutting member positioned at a distal location;

FIG. 7A depicts a side elevational view of an exemplary alternative end effector for incorporation into the device of FIG. 1, with jaws in an open position and a cutting member with a cable drive at a proximal location;

FIG. 7B depicts a side elevational view of the end effector of FIG. 7A, with jaws in a closed position and the cutting member at the proximal location;

FIG. 7C depicts a side elevational view of the end effector of FIG. 7A, with jaws in the closed position and the cutting member at a distal location;

FIG. 8 depicts a side elevational view of the blade of FIG. 7A;

FIG. 9 depicts a side elevational view of the jaw pivoting cable of FIG. 7A;

FIG. 10 depicts a top plan view of an articulation joint for use with the end effector of FIG. 7A;

FIG. 11A depicts a side elevational view of another exemplary end effector for incorporation into the device of FIG. 1, with jaws in the open position and a cutting member with a ball drive at a proximal location;

FIG. 11B depicts a side elevational view of the end effector of FIG. 11A, with jaws in a closed position and the cutting member at the proximal location;

FIG. 11C depicts a side elevational view of the end effector of FIG. 11A, with jaws in the closed position and the cutting member at the distal location;

FIG. 12 depicts a cross-sectional view of the end effector of FIG. 11C, taken along the line 12-12 of FIG. 11C;

FIG. 13 depicts a top plan view of an articulation joint for use with the end effector of FIG. 11A;

FIG. 14A depicts a partial perspective view of an exemplary alternative end effector for incorporation into the device of FIG. 1, with a cutting member positioned at a proximal location;

FIG. 14B depicts a partial perspective view of the end effector of FIG. 14A, with the cutting member positioned at a distal location;

FIG. 15A depicts a top plan view of the end effector of FIG. 14A, with the cutting member positioned at the proximal location;

FIG. 15B depicts a top plan view of the end effector of FIG. 14A, with the cutting member positioned at the distal location;

FIG. 16A depicts a side view of an exemplary alternative end effector for incorporation into the device of FIG. 1, with a rotary driven cutting member positioned at a proximal location;

FIG. 16B depicts a side view of the end effector of FIG. 16A, with the rotary driven cutting member positioned at a distal location;

FIG. 17A depicts a side view of an exemplary alternative end effector for incorporation into the device of FIG. 1, with a rotary driven cutting member positioned at the proximal location;

FIG. 17B depicts a side view of the end effector of FIG. 17A, with the rotary driven cutting member positioned at the distal location;

FIG. 18 depicts a perspective view of an exemplary alternative end effector for incorporation into the device of FIG. 1, with a gear assembly positioned in the articulation joint; and

FIG. 19 depicts a top partial view of the gear assembly of FIG. 18.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Exemplary Electrosurgical Device with Articulation Feature

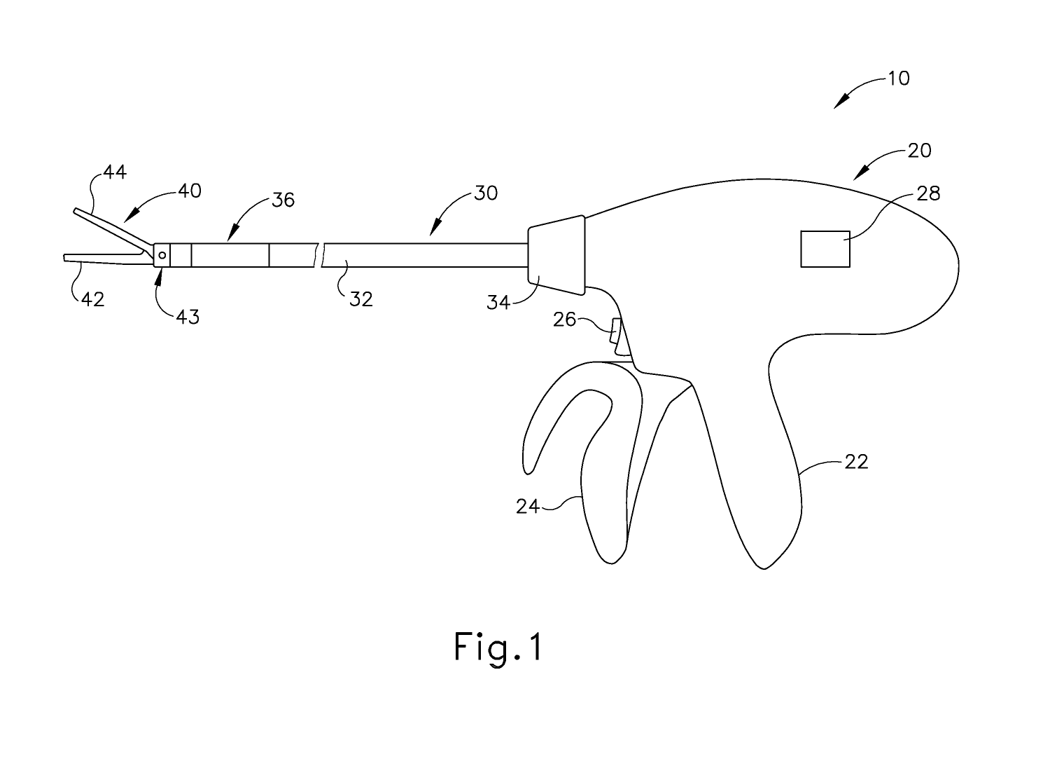

FIGS. 1-4 show an exemplary electrosurgical instrument (10) that is constructed and operable in accordance with at least some of the teachings of U.S. Pat. No. 6,500,176; U.S. Pat. No. 7,112,201; U.S. Pat. No. 7,125,409; U.S. Pat. No. 7,169,146; U.S. Pat. No. 7,186,253; U.S. Pat. No. 7,189,233; U.S. Pat. No. 7,220,951; U.S. Pat. No. 7,309,849; U.S. Pat. No. 7,311,709; U.S. Pat. No. 7,354,440; U.S. Pat. No. 7,381,209; U.S. Pub. No. 2011/0087218; and/or U.S. patent application Ser. No. 13/151,181. As described therein and as will be described in greater detail below, electrosurgical instrument (10) is operable to cut tissue and seal or weld tissue (e.g., a blood vessel, etc.) substantially simultaneously. In other words, electrosurgical instrument (10) operates similar to an endocutter type of stapler, except that electrosurgical instrument (10) provides tissue welding through application of bipolar RF energy instead of providing lines of staples to join tissue. It should also be understood that electrosurgical instrument (10) may have various structural and functional similarities with the ENSEAL® Tissue Sealing Device by Ethicon Endo-Surgery, Inc., of Cincinnati, Ohio. Furthermore, electrosurgical instrument (10) may have various structural and functional similarities with the devices taught in any of the other references that are cited and incorporated by reference herein. To the extent that there is some degree of overlap between the teachings of the references cited herein, the ENSEAL® Tissue Sealing Device by Ethicon Endo-Surgery, Inc., of Cincinnati, Ohio, and the following teachings relating to electrosurgical instrument (10), there is no intent for any of the description herein to be presumed as admitted prior art. Several teachings below will in fact go beyond the scope of the teachings of the references cited herein and the ENSEAL® Tissue Sealing Device by Ethicon Endo-Surgery, Inc., of Cincinnati, Ohio.

A. Exemplary Handpiece and Shaft

Electrosurgical instrument (10) of the present example includes a handpiece (20), a shaft (30) extending distally from handpiece (20), and an end effector (40) disposed at a distal end of shaft (30). Handpiece (20) of the present example includes a pistol grip (22), a pivoting trigger (24), an activation button (26), and an articulation control (28). Trigger (24) is pivotable toward and away from pistol grip (22) to selectively actuate end effector (40) as will be described in greater detail below. Activation button (26) is operable to selectively activate RF circuitry that is in communication with end effector (40), as will also be described in greater detail below. In some versions, activation button (26) also serves as a mechanical lockout against trigger (24), such that trigger (24) cannot be fully actuated unless button (26) is being pressed simultaneously. Examples of how such a lockout may be provided are disclosed in one or more of the references cited herein. It should be understood that pistol grip (22), trigger (24), and button (26) may be modified, substituted, supplemented, etc. in any suitable way, and that the descriptions of such components herein are merely illustrative. Articulation control (28) of the present example is operable to selectively control articulation section (36) of shaft (30), which will be described in greater detail below. Various examples of forms that articulation control (28) may take will also be described in greater detail below, while further examples will be apparent to those of ordinary skill in the art in view of the teachings herein.

Shaft (30) of the present example includes an outer sheath (32) and an articulating section (36). Articulating section (36) is operable to selectively position end effector (40) at various angles relative to the longitudinal axis defined by sheath (32). Various examples of forms that articulating section (36) and other components of shaft (30) may take will be described in greater detail below, while further examples will be apparent to those of ordinary skill in the art in view of the teachings herein. For instance, it should be understood that various components that are operable to actuate articulating section (36) may extend through the interior of sheath (32). In some versions, shaft (30) is also rotatable about the longitudinal axis defined by sheath (32), relative to handpiece (20), via a knob (34). Such rotation may provide rotation of end effector (40) and shaft (30) unitarily. In some other versions, knob (34) is operable to rotate end effector (40) without rotating any portion of shaft (30) that is proximal of articulating section (36). As another merely illustrative example, electrosurgical instrument (10) may include one rotation control that provides rotatability of shaft (30) and end effector (40) as a single unit; and another rotation control that provides rotatability of end effector (40) without rotating any portion of shaft (30) that is proximal of articulating section (36). Other suitable rotation schemes will be apparent to those of ordinary skill in the art in view of the teachings herein. Of course, rotatable features may simply be omitted if desired.

B. Exemplary End Effector

End effector (40) of the present example comprises a first jaw (42) and a second jaw (44). In the present example, second jaw (44) is substantially fixed relative to shaft (30); while first jaw (42) pivots relative to shaft (30), toward and away from second jaw (42). In some versions, actuators such as rods or cables, etc., may extend through sheath (32) and be joined with first jaw (42) at a pivotal coupling (43), such that longitudinal movement of the actuator rods/cables/etc. through shaft (30) provides pivoting of first jaw (42) relative to shaft (30) and relative to second jaw (44). Of course, jaws (42, 44) may instead have any other suitable kind of movement and may be actuated in any other suitable fashion. By way of example only, and as will be described in greater detail below, jaws (42, 44) may be actuated and thus closed by longitudinal translation of a firing beam (60), such that actuator rods/cables/etc. may simply be eliminated in some versions.

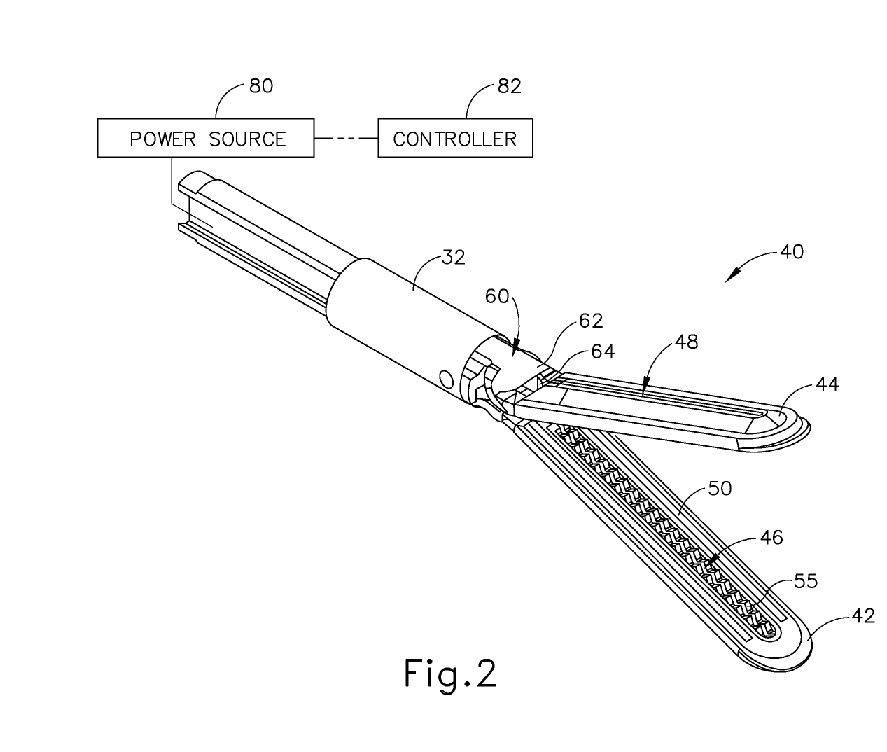

As best seen in FIGS. 2-4, first jaw (42) defines a longitudinally extending elongate slot (46); while second jaw (44) also defines a longitudinally extending elongate slot (48). In addition, the top side of first jaw (42) presents a first electrode surface (50); while the underside of second jaw (44) presents a second electrode surface (52). Electrode surfaces (50, 52) are in communication with an electrical source (80) via one or more conductors (not shown) that extend along the length of shaft (30). Electrical source (80) is operable to deliver RF energy to first electrode surface (50) at a first polarity and to second electrode surface (52) at a second (opposite) polarity, such that RF current flows between electrode surfaces (50, 52) and thereby through tissue captured between jaws (42, 44). In some versions, firing beam (60) serves as an electrical conductor that cooperates with electrode surfaces (50, 52) (e.g., as a ground return) for delivery of bipolar RF energy captured between jaws (42, 44). Electrical source (80) may be external to electrosurgical instrument (10) or may be integral with electrosurgical instrument (10) (e.g., in handpiece (20), etc.), as described in one or more references cited herein or otherwise. A controller (82) regulates delivery of power from electrical source (80) to electrode surfaces (50, 52). Controller (82) may also be external to electrosurgical instrument (10) or may be integral with electrosurgical instrument (10) (e.g., in handpiece (20), etc.), as described in one or more references cited herein or otherwise. It should also be understood that electrode surfaces (50, 52) may be provided in a variety of alternative locations, configurations, and relationships.

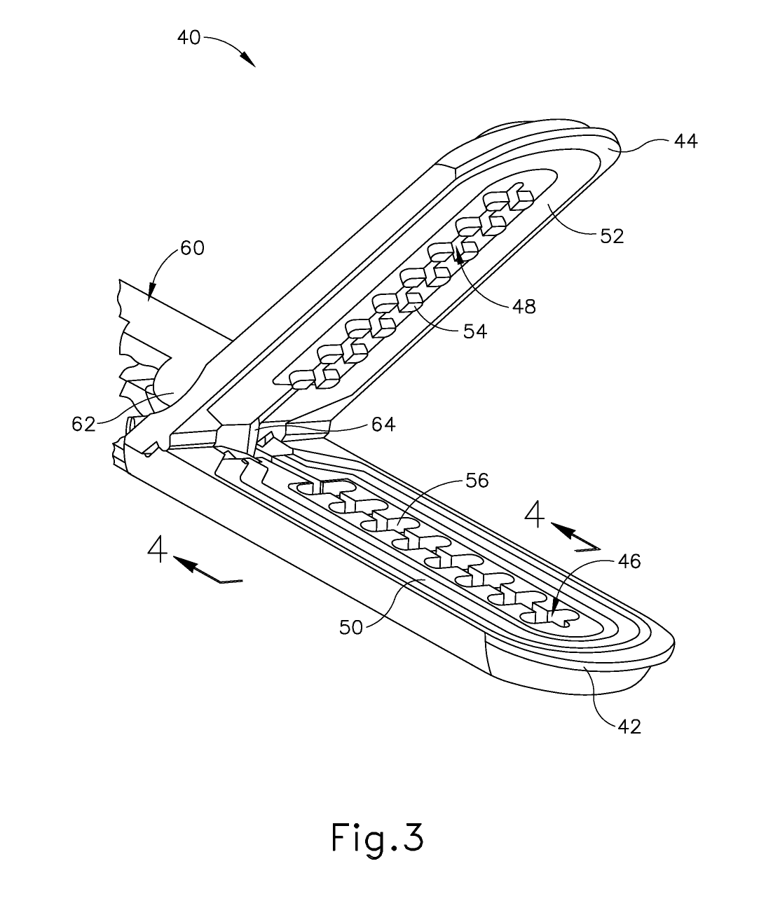

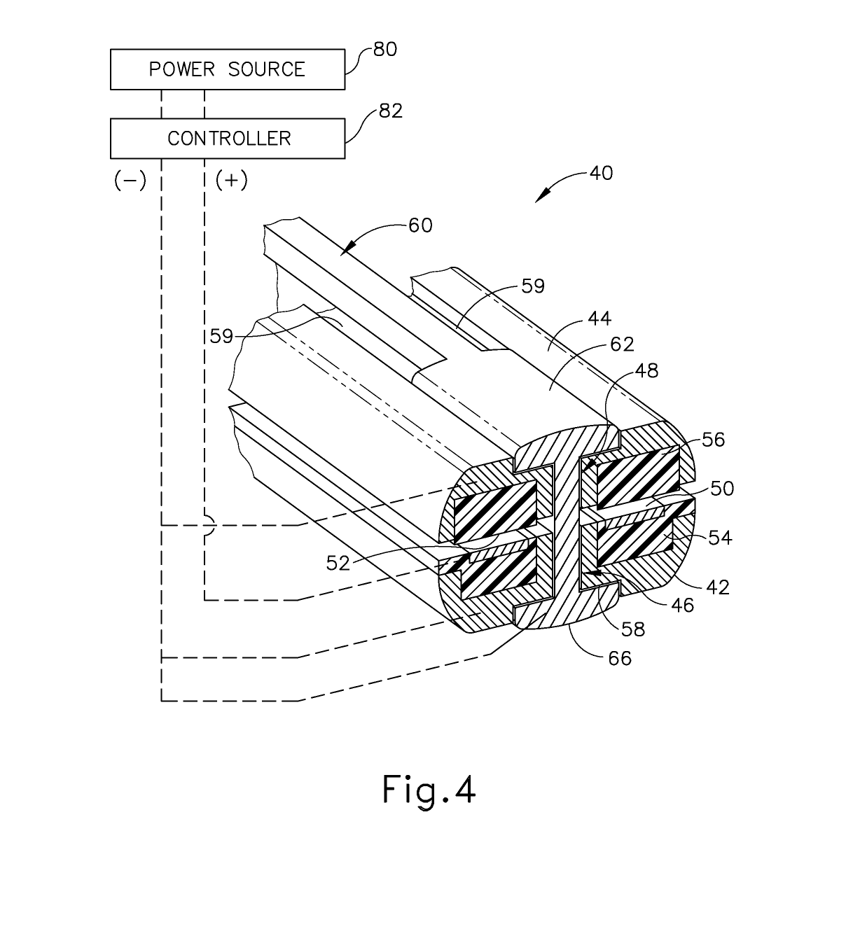

As best seen in FIG. 4, the lower side of first jaw (42) includes a longitudinally extending recess (58) adjacent to slot (46); while the upper side of second jaw (44) includes a longitudinally extending recess (59) adjacent to slot (48). FIG. 2 shows the upper side of first jaw (42) including a plurality of teeth serrations (46). It should be understood that the lower side of second jaw (44) may include complementary serrations that nest with serrations (46), to enhance gripping of tissue captured between jaws (42, 44) without necessarily tearing the tissue. FIG. 3 shows an example of serrations (46) in first jaw (42) as mainly recesses; with serrations (48) in second jaw (44) as mainly protrusions. Of course, serrations (46, 48) may take any other suitable form or may be simply omitted altogether. It should also be understood that serrations (46, 48) may be formed of an electrically non-conductive, or insulative, material, such as plastic, glass, and/or ceramic, for example, and may include a treatment such as polytetrafluoroethylene, a lubricant, or some other treatment to substantially prevent tissue from getting stuck to jaws (42, 44).

With jaws (42, 44) in a closed position, shaft (30) and end effector (40) are sized and configured to fit through trocars having various inner diameters, such that electrosurgical instrument (10) is usable in minimally invasive surgery, though of course electrosurgical instrument (10) could also be used in open procedures if desired. By way of example only, with jaws (42, 44) in a closed position, shaft (30) and end effector (40) may present an outer diameter of approximately 5 mm. Alternatively, shaft (30) and end effector (40) may present any other suitable outer diameter (e.g., between approximately 2 mm and approximately 20 mm, etc.).

As another merely illustrative variation, either jaw (42, 44) or both of jaws (42, 44) may include at least one port, passageway, conduit, and/or other feature that is operable to draw steam, smoke, and/or other gases/vapors/etc. from the surgical site. Such a feature may be in communication with a source of suction, such as an external source or a source within handpiece (20), etc. In addition, end effector (40) may include one or more tissue cooling features (not shown) that reduce the degree or extent of thermal spread caused by end effector (40) on adjacent tissue when electrode surfaces (50, 52) are activated. Various suitable forms that such cooling features may take will be apparent to those of ordinary skill in the art in view of the teachings herein.

In some versions, end effector (40) includes one or more sensors (not shown) that are configured to sense a variety of parameters at end effector (40), including but not limited to temperature of adjacent tissue, electrical resistance or impedance of adjacent tissue, voltage across adjacent tissue, forces exerted on jaws (42, 44) by adjacent tissue, etc. By way of example only, end effector (40) may include one or more positive temperature coefficient (PTC) thermistor bodies (54, 56) (e.g., PTC polymer, etc.), located adjacent to electrodes (50, 52) and/or elsewhere. Data from sensors may be communicated to controller (82). Controller (82) may process such data in a variety of ways. By way of example only, controller (82) may modulate or otherwise change the RF energy being delivered to electrode surfaces (50, 52), based at least in part on data acquired from one or more sensors at end effector (40). In addition or in the alternative, controller (82) may alert the user to one or more conditions via an audio and/or visual feedback device (e.g., speaker, lights, display screen, etc.), based at least in part on data acquired from one or more sensors at end effector (40). It should also be understood that some kinds of sensors need not necessarily be in communication with controller (82), and may simply provide a purely localized effect at end effector (40). For instance, a PTC thermistor bodies (54, 56) at end effector (40) may automatically reduce the energy delivery at electrode surfaces (50, 52) as the temperature of the tissue and/or end effector (40) increases, thereby reducing the likelihood of overheating. In some such versions, a PTC thermistor element is in series with power source (80) and electrode surface (50, 52); and the PTC thermistor provides an increased impedance (reducing flow of current) in response to temperatures exceeding a threshold. Furthermore, it should be understood that electrode surfaces (50, 52) may be used as sensors (e.g., to sense tissue impedance, etc.). Various kinds of sensors that may be incorporated into electrosurgical instrument (10) will be apparent to those of ordinary skill in the art in view of the teachings herein. Similarly various things that can be done with data from sensors, by controller (82) or otherwise, will be apparent to those of ordinary skill in the art in view of the teachings herein. Other suitable variations for end effector (40) will also be apparent to those of ordinary skill in the art in view of the teachings herein.

C. Exemplary Firing Beam

As also seen in FIGS. 2-4, electrosurgical instrument (10) of the present example includes a firing beam (60) that is longitudinally movable along part of the length of end effector (40). Firing beam (60) is coaxially positioned within shaft (30), extends along the length of shaft (30), and translates longitudinally within shaft (30) (including articulating section (36) in the present example), though it should be understood that firing beam (60) and shaft (30) may have any other suitable relationship. Firing beam (60) includes a sharp distal blade (64), an upper flange (62), and a lower flange (66). As best seen in FIG. 4, distal blade (64) extends through slots (46, 48) of jaws (42, 44), with upper flange (62) being located above jaw (44) in recess (59) and lower flange (66) being located below jaw (42) in recess (58). The configuration of distal blade (64) and flanges (62, 66) provides an “I-beam” type of cross section at the distal end of firing beam (60). While flanges (62, 66) extend longitudinally only along a small portion of the length of firing beam (60) in the present example, it should be understood that flanges (62, 66) may extend longitudinally along any suitable length of firing beam (60). In addition, while flanges (62, 66) are positioned along the exterior of jaws (42, 44), flanges (62, 66) may alternatively be disposed in corresponding slots formed within jaws (42, 44). For instance, each jaw (42, 44) may define a “T”-shaped slot, with parts of distal blade (64) being disposed in one vertical portion of each “T”-shaped slot and with flanges (62, 66) being disposed in the horizontal portions of the “T”-shaped slots. Various other suitable configurations and relationships will be apparent to those of ordinary skill in the art in view of the teachings herein.

Distal blade (64) is substantially sharp, such that distal blade will readily sever tissue that is captured between jaws (42, 44). Distal blade (64) is also electrically grounded in the present example, providing a return path for RF energy as described elsewhere herein. In some other versions, distal blade (64) serves as an active electrode. In addition or in the alternative, distal blade (64) may be selectively energized with ultrasonic energy (e.g., harmonic vibrations at approximately 55.5 kHz, etc.).

The “I-beam” type of configuration of firing beam (60) provides closure of jaws (42, 44) as firing beam (60) is advanced distally. In particular, flange (62) urges jaw (44) pivotally toward jaw (42) as firing beam (60) is advanced from a proximal position (FIGS. 1-3) to a distal position (FIG. 4), by bearing against recess (59) formed in jaw (44). This closing effect on jaws (42, 44) by firing beam (60) may occur before distal blade (64) reaches tissue captured between jaws (42, 44). Such staging of encounters by firing beam (60) may reduce the force required to squeeze grip (24) to actuate firing beam (60) through a full firing stroke. In other words, in some such versions, firing beam (60) may have already overcome an initial resistance required to substantially close jaws (42, 44) on tissue before encountering resistance from the tissue captured between jaws (42, 44). Of course, any other suitable staging may be provided.

In the present example, flange (62) is configured to cam against a ramp feature at the proximal end of jaw (44) to open jaw (42) when firing beam (60) is retracted to a proximal position and to hold jaw (42) open when firing beam (60) remains at the proximal position. This camming capability may facilitate use of end effector (40) to separate layers of tissue, to perform blunt dissections, etc., by forcing jaws (42, 44) apart from a closed position. In some other versions, jaws (42, 44) are resiliently biased to an open position by a spring or other type of resilient feature. While jaws (42, 44) close or open as firing beam (60) is translated in the present example, it should be understood that other versions may provide independent movement of jaws (42, 44) and firing beam (60). By way of example only, one or more cables, rods, beams, or other features may extend through shaft (30) to selectively actuate jaws (42, 44) independently of firing beam (60). Such jaw (42, 44) actuation features may be separately controlled by a dedicated feature of handpiece (20). Alternatively, such jaw actuation features may be controlled by trigger (24) in addition to having trigger (24) control firing beam (60). It should also be understood that firing beam (60) may be resiliently biased to a proximal position, such that firing beam (60) retracts proximally when a user relaxes their grip on trigger (24).

D. Exemplary Operation

In an exemplary use, end effector (40) is inserted into a patient via a trocar.

Articulation section (36) is substantially straight when end effector (40) and part of shaft (30) are inserted through the trocar. Articulation control (28) may then be manipulated to pivot or flex articulation section (36) of shaft (30) in order to position end effector (40) at a desired position and orientation relative to an anatomical structure within the patient. Two layers of tissue of the anatomical structure are then captured between jaws (42, 44) by squeezing trigger (24) toward pistol grip (22). Such layers of tissue may be part of the same natural lumen defining anatomical structure (e.g., blood vessel, portion of gastrointestinal tract, portion of reproductive system, etc.) in a patient. For instance, one tissue layer may comprise the top portion of a blood vessel while the other tissue layer may comprise the bottom portion of the blood vessel, along the same region of length of the blood vessel (e.g., such that the fluid path through the blood vessel before use of electrosurgical instrument (10) is perpendicular to the longitudinal axis defined by end effector (40), etc.). In other words, the lengths of jaws (42, 44) may be oriented perpendicular to (or at least generally transverse to) the length of the blood vessel. As noted above, flanges (62, 66) cammingly act to pivot jaw (44) toward jaw (44) when firing beam (60) is actuated distally by squeezing trigger (24) toward pistol grip (22).

With tissue layers captured between jaws (42, 44) firing beam (60) continues to advance distally by the user squeezing trigger (24) toward pistol grip (22). As firing beam (60) advances distally, distal blade (64) simultaneously severs the clamped tissue layers, resulting in separated upper layer portions being apposed with respective separated lower layer portions. In some versions, this results in a blood vessel being cut in a direction that is generally transverse to the length of the blood vessel. It should be understood that the presence of flanges (62, 66) immediately above and below jaws (42, 44), respectively, may help keep jaws (42, 44) in a closed and tightly clamping position. In particular, flanges (62, 66) may help maintain a significantly compressive force between jaws (42, 44). With severed tissue layer portions being compressed between jaws (42, 44), electrode surfaces (50, 52) are activated with bipolar RF energy by the user depressing activation button (26). In some versions, electrodes (50, 52) are selectively coupled with power source (80) (e.g., by the user depressing button (26), etc.) such that electrode surfaces (50, 52) of jaws (42, 44) are activated with a common first polarity while firing beam (60) is activated at a second polarity that is opposite to the first polarity. Thus, a bipolar RF current flows between firing beam (60) and electrode surfaces (50, 52) of jaws (42, 44), through the compressed regions of severed tissue layer portions. In some other versions, electrode surface (50) has one polarity while electrode surface (52) and firing beam (60) both have the other polarity. In either version (among at least some others), bipolar RF energy delivered by power source (80) ultimately thermally welds the tissue layer portions on one side of firing beam (60) together and the tissue layer portions on the other side of firing beam (60) together.

In certain circumstances, the heat generated by activated electrode surfaces (50, 52) can denature the collagen within the tissue layer portions and, in cooperation with clamping pressure provided by jaws (42, 44), the denatured collagen can form a seal within the tissue layer portions. Thus, the severed ends of the natural lumen defining anatomical structure are hemostatically sealed shut, such that the severed ends will not leak bodily fluids. In some versions, electrode surfaces (50, 52) may be activated with bipolar RF energy before firing beam (60) even begins to translate distally and thus before the tissue is even severed. For instance, such timing may be provided in versions where button (26) serves as a mechanical lockout relative to trigger (24) in addition to serving as a switch between power source (80) and electrode surfaces (50, 52).

While several of the teachings below are described as variations to electrosurgical instrument (10), it should be understood that various teachings below may also be incorporated into various other types of devices. By way of example only, in addition to being readily incorporated into electrosurgical instrument (10), various teachings below may be readily incorporated into the devices taught in any of the references cited herein, other types of electrosurgical devices, surgical staplers, surgical clip appliers, and tissue graspers, among various other devices. Other suitable devices into which the following teachings may be incorporated will be apparent to those of ordinary skill in the art in view of the teachings herein.

II. Exemplary Articulation Joint Configurations

As noted above, some versions of shaft (30) include an articulation section (36), which is operable to selectively position end effector (40) at various angles relative to the longitudinal axis defined by sheath (32). Several examples of forms that articulation section (36) and other components of shaft (30) may take will be described in greater detail below, while further examples will be apparent to those of ordinary skill in the art in view of the teachings herein. By way of example only, some merely illustrative alternative examples of articulation section (36) are disclosed in U.S. Pub. No. 2012/0078248, entitled “Articulation Joint Features for Articulating Surgical Device,” published Mar. 29, 2012, the disclosure of which is incorporated by reference herein.

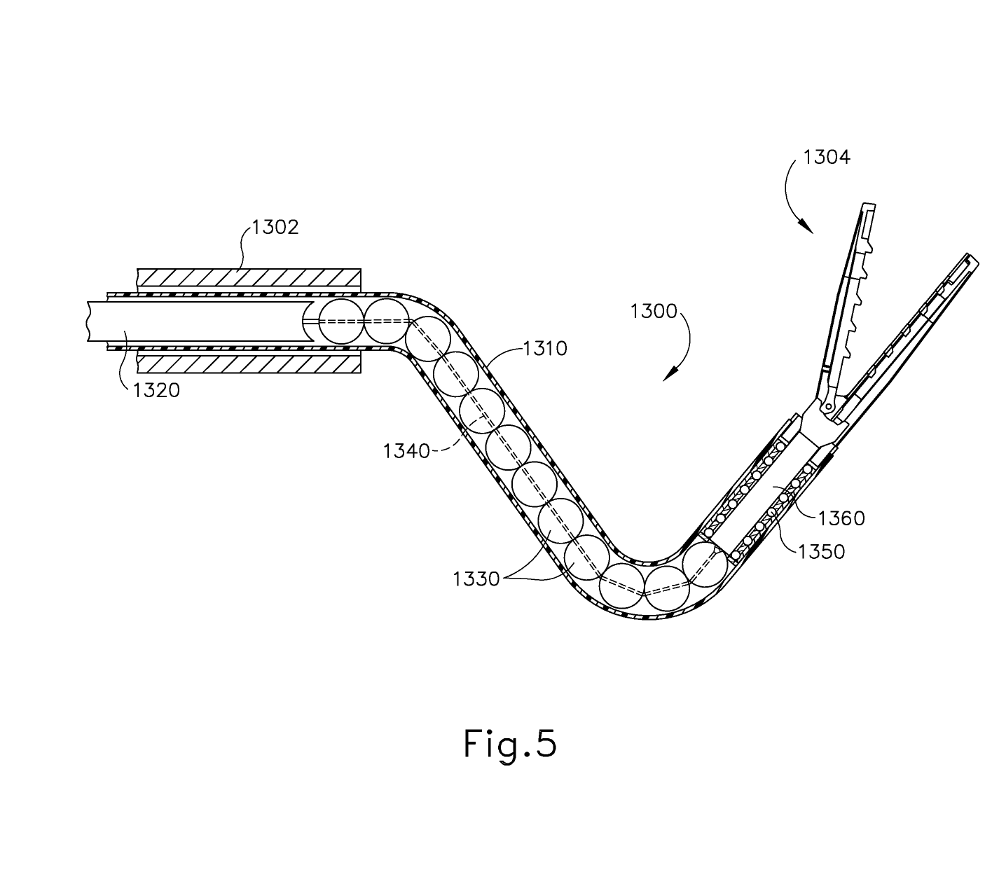

FIG. 5 shows exemplary components and configurations that may be used to actuate a firing beam through a pre-bent articulation section (1300). In this example, articulation section (1300) includes sheath (1310) that is preformed to include a pair of bends that position an end effector (1304) at an angle relative to a rigid shaft section (1302). It should be understood that these features may be readily incorporated into electrosurgical instrument (10) described above, with shaft section (1302) corresponding to shaft (30) and end effector (1304) corresponding to end effector (40). In some versions (1310), sheath (1310) is resiliently biased to assume the configuration shown in FIG. 5, but may be selectively straightened in order to pass through a trocar or other cannula in order to reach the interior of a patient in a minimally invasive manner.

In the example shown in FIG. 5, a push rod (1320) is slidably positioned within a proximal portion of sheath (1310). A plurality of bearings (1330) are positioned adjacent to one another within sheath (1310), distal to push rod (1320). A wire (1340) passes through bearings (1330), such that bearings (1330) are tethered together by wire (1340). Wire (1340) is configured to communicate power from a power source to end effector (1304). Bearings (1330) are configured to translate within sheath (1310) and are thus operable to transmit distal translational motion from push rod (1320) to firing beam (1360). Firing beam (1360) is thereby operable in accordance with firing beam (60) as described above. In the present example, bearings (1330) translate along the bent path formed by sheath (1310) without straightening sheath when bearings (1330) are advanced distally. Wire (1340) is structurally coupled to firing beam (1360) and push rod (1320) such that wire (1340) is operable to retract firing beam (1360) proximally when push rod (1320) is retracted proximally. Thus, end effector (1340) may be opened by retracting push rod. A set of bearings (1350) are used support firing beam (1360) in this example, though it should be understood that various alternative structures may be used.

Articulation section (36) of shaft (30) may take a variety of forms. By way of example only, articulation section (36) may be configured in accordance with one or more teachings of U.S. Pub. No. 2012/0078247, entitled “Articulation Joint Features for Articulating Surgical Device,” published Mar. 29, 2012, the disclosure of which is incorporated by reference herein. As another merely illustrative example, articulation section (36) may be configured in accordance with one or more teachings of U.S. Pub. No. 2012/0078248, entitled “Articulation Joint Features for Articulating Surgical Device,” published Mar. 29, 2012, the disclosure of which is incorporated by reference herein. Furthermore, articulation section may be configured in accordance with the teachings of at least one other of the references cited herein. Articulation section (36) may provide articulation angles of greater than about 45°, greater than about 90°, or greater than about 135°. Various other suitable forms that articulation section (36) may take will be apparent to those of ordinary skill in the art in view of the teachings herein.

III. Exemplary Articulation Control Configurations

Articulation control (28) may take a variety of forms. By way of example only, articulation control (28) may be configured in accordance with one or more teachings of U.S. Pub. No. 2012/0078243, entitled “Control Features for Articulating Surgical Device,” published on Mar. 29, 2012, the disclosure of which is incorporated by reference herein. As another merely illustrative example, articulation control (28) may be configured in accordance with one or more teachings of U.S. Pub. No. 2012/0078244, entitled “Control Features for Articulating Surgical Device,” published on Mar. 29, 2012, the disclosure of which is incorporated by reference herein. Furthermore, articulation section may be configured in accordance with the teachings of at least one other of the references cited herein. Various other suitable forms that articulation control (28) may take will be apparent to those of ordinary skill in the art in view of the teachings herein.

IV. Exemplary Cutting Member Actuation Features

It should be understood that any of the versions of electrosurgical instrument (10) described herein may include various other features in addition to or in lieu of those described above. The following examples provide features for driving blade (64) while articulation section (36) is significantly articulated. The following examples also enable articulation section (36) to be substantially close to jaws (42, 44) of end effector (40), which may further facilitate access to otherwise difficult surgical sites. Several examples of such other features are described below, while other features will be apparent to those of ordinary skill in the art in view of the teachings herein. It should be understood that the following examples may be used in versions of electrosurgical device (10) that lack an articulation section (36). It should also be understood that, just like various other components described herein, the following examples may be used in a variety of other types of devices beyond electrosurgical devices, including but not limited to endocutter surgical stapling devices. Other suitable implementations will be apparent to those of ordinary skill in the art in view of the teachings herein.

A. Exemplary Firing Band Actuation

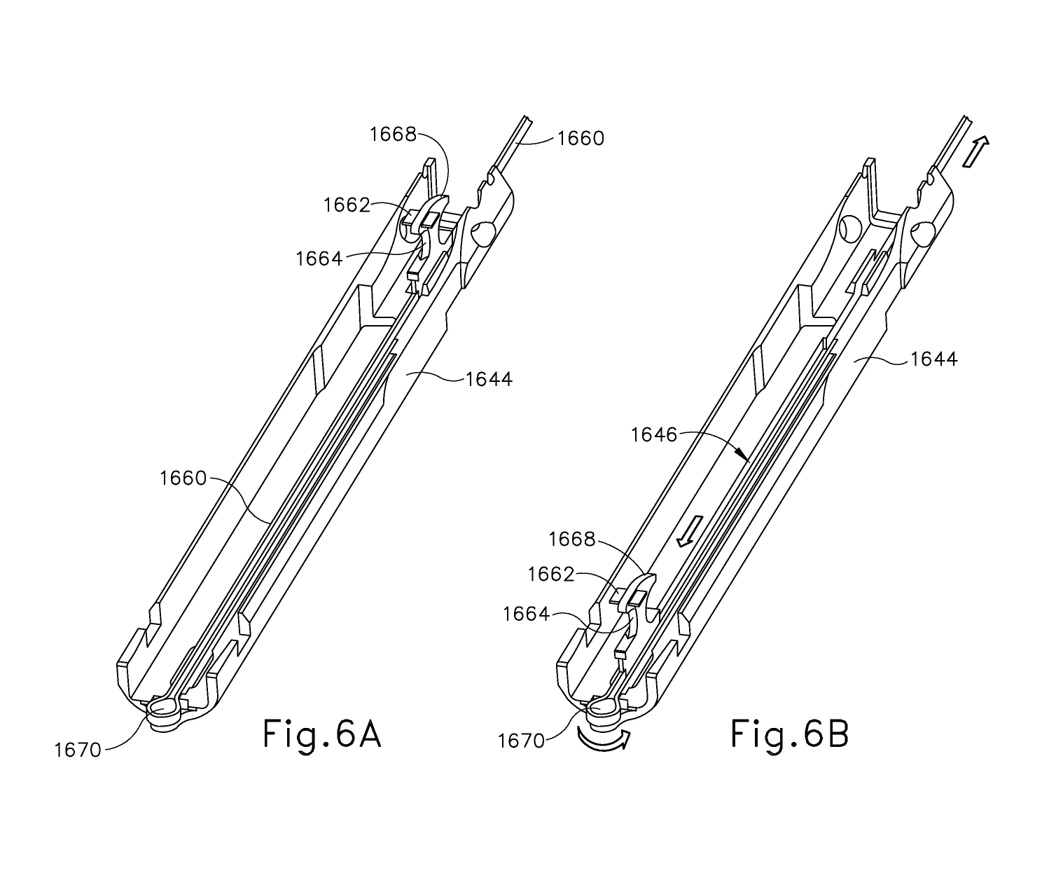

In examples described above, a blade (64) is advanced distally through end effector (40) by advancing firing bar (60) distally. In the example depicted in FIGS. 6A-6B, a blade (1664) is advanced distally by retracting a firing band (1660) proximally. In this example, blade (1664) is distally presented by a blade member (1668), which includes upper flange members (1662) and which is secured to a distal end of firing band (1660). Blade member (1668) travels longitudinally along a slot (1646) formed in a lower jaw (1644) of an end effector assembly. Blade member (1668) further includes lower flange members (not shown) that are disposed beneath lower jaw (1644). By way of example only, lower jaw (44) of electrosurgical instrument (10) may be readily modified to include the features of lower jaw (1644) described in this example.

The distal end of lower jaw (1644) includes a post (1670). Firing band (1660) is wrapped around post (1670) such that post (1670) redirects the longitudinal motion of firing band (1660) by approximately 180°. Firing band (1660) has sufficient flexibility to provide such motion, yet also has enough tensile strength to bear significant loads on blade member (1668) as blade (1664) severs tissue. In some versions, post (1670) includes a bushing or bearing that is configured to facilitate movement of firing band (1660) about post (1670). As can be seen from the transition between FIG. 6A and FIG. 6B, blade member (1668) advances distally along channel (1646) from a proximal position to a distal position in response to proximal movement of firing band (1660). In some versions, firing band (1660) may also be advanced distally to return blade member (1668) from the distal position to the proximal position. For instance, lower jaw (1644) may include guide channels that guide firing band (1660) and prevent firing band (1660) from buckling as firing band (1660) is advanced distally. Various suitable ways in which firing band (1660) may be translated distally and/or proximally will be apparent to those of ordinary skill in the art in view of the teachings herein. It should also be understood that, in some versions, advancing blade member (1668) distally by pulling firing band (1660) proximally when the end effector is articulated may be relatively easier than advancing a blade member (1668) would otherwise be if a firing beam (60) were advanced distally to advance a blade member with an end effector (40) articulated. In other words, firing band (1660) may facilitate configurations with articulation angles that are greater than those provided by devices that use distally advanced firing beams (60).

B. Exemplary Closure and Cutting Member Actuation

Instead of a firing band (1660), a blade (64) may be advanced through an end effector (40) using other features, such as a cable or a plurality of bearings. Several examples of such other features are described below, while other features will be apparent to those of ordinary skill in the art in view of the teachings herein.

### 1. Exemplary Cable Actuation

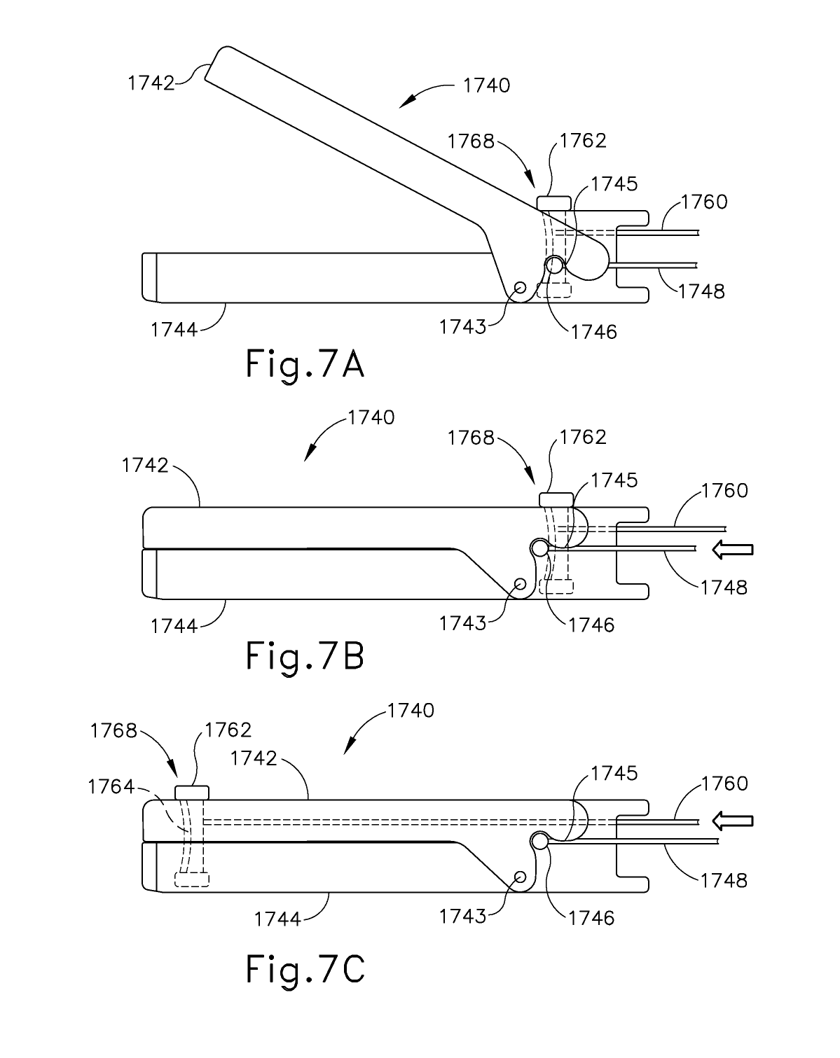

FIGS. 7A-10 show an exemplary end effector (1740) in which a blade member (1768) may be advanced through the end effector (1740) using a blade cable (1760). End effector (1740) is similar to end effector (40), except that upper jaw (1742) provides a modified pivoting action, as shown in FIGS. 7A-7C. In particular, upper jaw (1742) comprises a ramped surface (1745) extending proximally from upper jaw (1742). Ramped surface (1745) is curved to correspond to a sphere (1746) that is fixed to a distal end of a jaw cable (1748), as shown in greater detail in FIG. 9. Although a sphere is shown in FIG. 9, sphere (1746) may be configured as other suitable shapes to correspond to ramped surface (1745).

Upper jaw (1742) pivots relative to lower jaw (1744) via pivotal coupling (1743) positioned distal to ramped surface (1745) by actuating jaw cable (1748). For example, jaw cable (1748) may be pushed distally to close upper jaw (1742) relative to lower jaw (1744) and jaw cable (1748) may be pulled proximally to open upper jaw (1742) relative to lower jaw (1744). Jaw cable (1748) may be used to substantially close jaws (1742, 1744) such that jaws (1742, 1744) are closed to about 90% compression or more. Jaw cable (1748) is configured to be rigid enough to translate upper jaw (1742), yet flexible enough to bend through a significantly articulated articulation section (36). One jaw cable (1748) may be provided on either side of end effector (1740), or two jaw cables (1748) may be provided on each side of end effector (1740). The proximal end of jaw cable (1748) may be coupled to trigger (24) of handpiece (20) such that when trigger (24) is squeezed and/or released, trigger (24) translates jaw cable (1748) to open and/or close jaws (1742, 1744). Jaw cable (1748) may also have a separate actuator on handpiece (20) to translate jaw cable (1748). For example, handpiece (20) may comprise a switch to translate jaw cable (1748) when the switch is moved proximally and/or distally. Suitable jaw cable (1748) configurations will be apparent to one with ordinary skill in the art in view of the teachings herein.

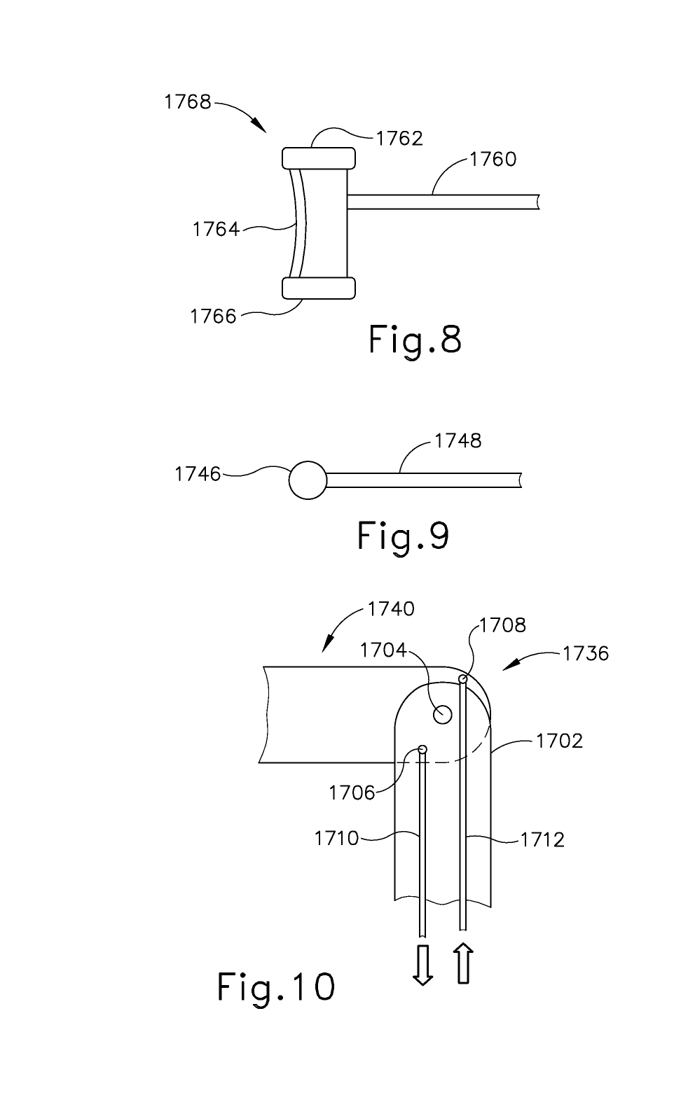

As shown in FIG. 8, blade member (1768) is similar to blade member (1668). Blade member (1768) comprises an upper flange (1762), a lower flange (1766), and a blade (1764) extending between upper flange (1762) and lower flange (1766). Upper flange (1762) is positioned on a top surface of upper jaw (1742). Lower jaw (1744) comprises a slot (not shown) similar to slot (1646) such that blade member (1768) may travel longitudinally along the slot. In particular, lower flange (1766) is positioned in the slot along lower jaw (1744). Instead of being translated by an I-beam (60), blade member (1768) is translated by a blade cable (1760). Blade cable (1760) extends proximally from blade member (1768). Blade cable (1760) is configured to translate blade member (1768) proximally and/or distally when blade cable (1760) is pushed and/or pulled. For example, blade cable (1760) may be pushed distally to translate blade member (1768) distally along jaws (1742, 1744) and blade cable (1760) may be pulled proximally to translate blade member (1768) proximally along the jaws (1742, 1744). As blade member (1768) travels distally, blade member (1760) travels along jaws (1742, 1744) to apply additional compression between jaws (1742, 1744) that was provided by jaw cable (1748). Blade (1764) severs tissue captured between jaws (1742, 1744) as blade (1764) travels distally. Blade cable (1760) is configured to be rigid enough to translate blade member (1768) through tissue, yet flexible enough to bend through a significantly articulated articulation section (36). The proximal end of blade cable (1760) may be coupled to trigger (24) of handpiece (20) such that when trigger (24) is squeezed and/or released, trigger (24) translates blade cable (1760). Blade cable (1760) may also have a separate actuator on handpiece (20) to translate blade cable (1760). For example, handpiece (20) may comprise a switch to translate blade cable (1760) when the switch is moved proximally and/or distally. Suitable blade cable (1760) configurations will be apparent to one with ordinary skill in the art in view of the teachings herein.

FIG. 10 shows an exemplary articulation section (1736) that may be coupled with end effector (1740). Articulation section (1736) is similar to articulation section (36). Articulation section (1736) comprises a pivot coupling (1704) that joins end effector (1740) with shaft (1702). Articulation cables (1710, 1712) extend through shaft (1702) and are secured to end effector (1740) via pins (1706, 1708). Cables (1710, 1712) are secured to end effector (1740) on opposing sides such that cables (1710, 1712) are pushed and/or pulled to articulate end effector (1740). For example, cable (1710) is pulled proximally and/or cable (1712) is pushed distally to articulate end effector (1740) to the left. Cable (1710) is pushed distally and/or cable (1712) is pulled proximally to articulate end effector (1740) to the right. Alternatively, only cable (1710, 1712) may be translated to articulate end effector (1740). While two cables (1710, 1712) are shown, a various number of other cables may be used. Articulation section (1736) may be positioned near jaws (1742, 1744) of end effector (1740). Articulation section (1736) may provide significant articulation angles of greater than about 45°, greater than about 90°, or greater than about 135°. Handpiece (20) may include an actuator to translate cables (1710, 1712). For example, handpiece (20) may comprise a switch for each cable (1710, 1712) to translate cables (1710, 1712) when each switch is moved proximally and/or distally. Cables (1710, 1712) may also be coupled to a rotation knob on handpiece (20) to translate cables (1710, 1712). For instance, if the rotation knob is rotated clockwise, cable (1712) may translate proximally and cable (1710) may translate distally. If the rotation knob is rotated counterclockwise, cable (1712) may translate distally and cable (1710) may translate proximally. By way of example only, cables (1710, 1712) may be actuated in accordance with at least some of the teachings of U.S. Pub. No. 2012/0078243, the disclosure of which is incorporated by reference herein; and/or U.S. Pub. No. 2012/0078244, the disclosure of which is incorporated by reference herein. Other suitable cable (1710, 1712) actuation configurations will be apparent to one with ordinary skill in the art in view of the teachings herein.

In an exemplary use, end effector (1740) is inserted into a patient via a trocar. Articulation section (1736) is substantially straight when end effector (1740) and part of shaft (1702) are inserted through the trocar, such that end effector (1740) is substantially aligned with shaft (1702). Articulation control (28) may then be manipulated to pivot or flex articulation section (1736) of shaft (1702) by pulling or pushing articulation cables (1710, 1712) in order to position end effector (1740) at a desired position and orientation relative to an anatomical structure within the patient. Two layers of tissue of the anatomical structure are then captured between jaws (1742, 1744) by squeezing trigger (24) toward pistol grip (22). Squeezing trigger (24) pushes jaw cable (1748). As jaw cable (1748) translates distally, ramp surface (1745) of upper jaw (1742) slides along sphere (1746) of jaw cable (1748) to substantially close jaws (1742, 1744) by pivoting jaw (1742) relative to jaw (1744), as shown in FIG. 7B. Squeezing trigger (24) also translates blade cable (1760) distally to translate blade member (1768) distally. Actuation of jaw cable (1748) and blade cable (1760) may be staged (e.g., trigger (24) may be squeezed through a first range of motion to actuate jaw cable (1748) to close jaws (1742, 1744), then trigger (24) may be squeezed through a second range of motion to actuate blade cable (1760) to drive blade (1764)). Alternatively, separate actuators may be provided on handpiece (20) to independently actuate jaw cable (1748) and blade cable (1760). Flanges (1762, 1766) cammingly act to close jaws (1742, 1744) further.

With tissue layers captured between jaws (1742, 1744) blade cable (1760) continues to advance distally by the user squeezing trigger (24) toward pistol grip (22), as shown in FIG. 7C. As blade cable (1760) advances distally, distal blade (1764) simultaneously severs the clamped tissue layers, resulting in separated upper layer portions being apposed with respective separated lower layer portions. Flanges (1762, 1766) help maintain a significantly compressive force between jaws (1742, 1744). With severed tissue layer portions being compressed between jaws (1742, 1744), electrode surfaces (50, 52) are activated with bipolar RF energy by the user depressing activation button (26). Bipolar RF energy delivered by power source (80) ultimately thermally welds the tissue layer portions on one side of blade member (1768) together and the tissue layer portions on the other side of blade member (1768) together. After the tissue layer portions have been welded, blade cable (1760) may be translated proximally to retract blade member (1768). In some versions, the tissue layer portions are welded prior to being severed. Jaw cable (1748) may be translated proximally to pivot jaw (1742) relative to jaw (1744) to open jaws (1742, 1744). Articulation cables (1710, 1712) may be pushed and/or pulled to pivot articulation section (1736) to reposition end effector (1740) to weld another layer of tissue or to remove end effector (1740) from the patient.

### 2. Exemplary Bearing Actuation

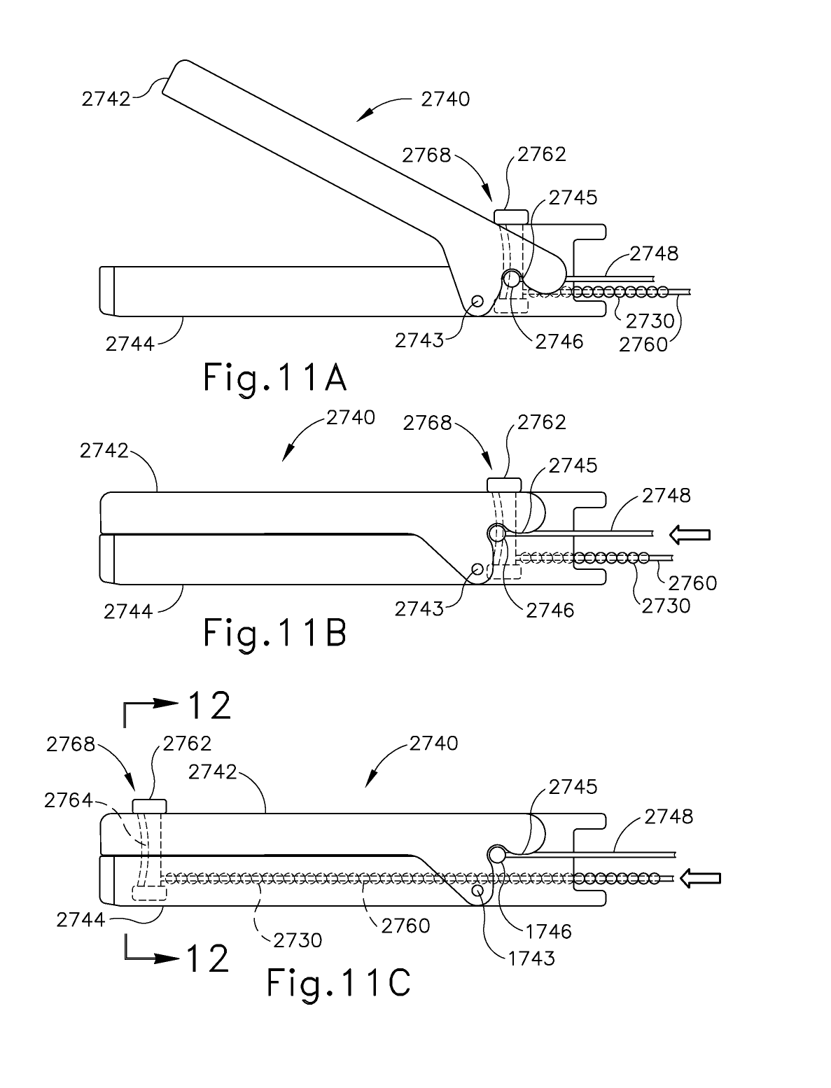

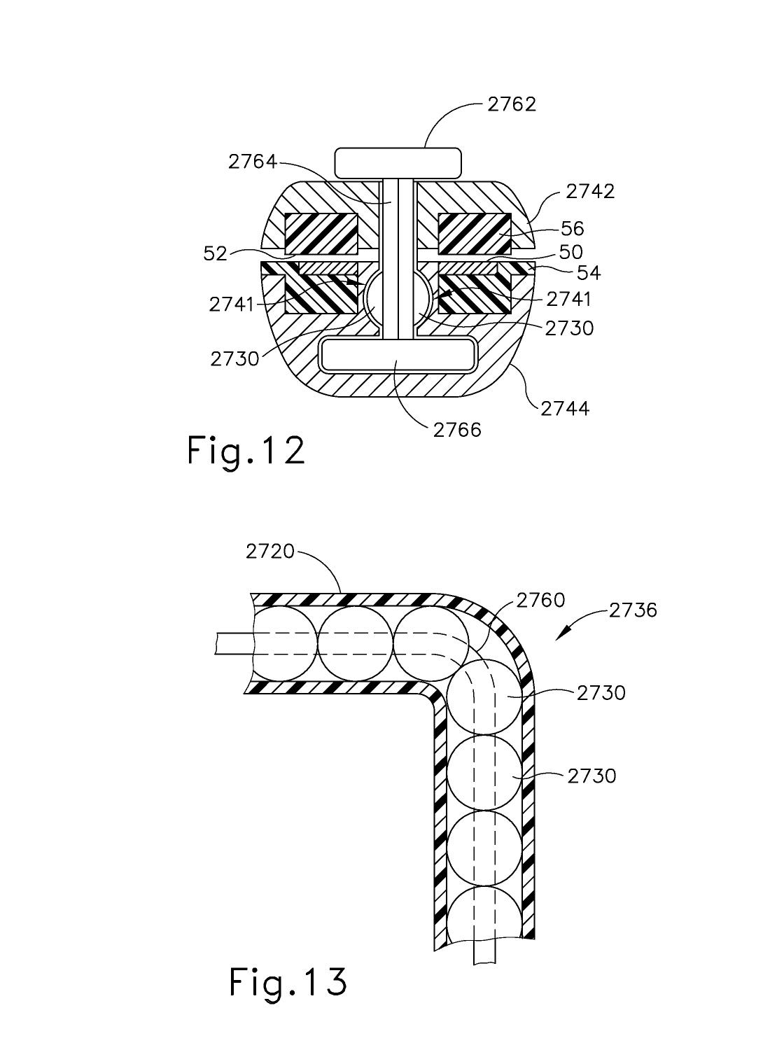

FIGS. 11A-13 show another exemplary end effector (2740) in which a plurality of bearings (2730) are used to translate blade member (2768) instead of a blade cable (1760). End effector (2740) is similar to end effector (1740), except that lower jaw (1744) comprises a modified blade member (2768) actuation. As shown in FIG. 12, blade member (2768) is similar to blade member (1768), except blade member (2768) is translated by a plurality of bearings (2730). Bearings (2730) are positioned proximal to blade member (2768). A wire (2760) extends through bearings (2730) and is secured to blade member (2768). A channel (2741) in lower jaw (2744) is configured to retain bearings (2730). Bearings (2730) and wire (2760) are configured to translate blade member (2768) proximally and/or distally when bearings (2730) and wire (2760) are pushed and/or pulled. For example, bearings (2730) may be pushed distally to translate blade member (2768) distally along channel (2741) in lower jaw (2744) and wire (2760) may be pulled proximally to translate blade member (2768) proximally along channel (2741) in lower jaw (2744).

FIG. 13 shows an exemplary bearing containment tube (2720). Containment tube (2720) of this example would be positioned within an articulation section, such as articulation sections (36, 1736) described above. Containment tube (2720) is in communication with end effector (2740), and is configured to contain bearings (2730) and wire (2760) through the articulation section. Containment tube (2720) may be flexible to accommodate articulation angles of greater than about 45°, greater than about 90°, or greater than about 135°. Containment tube (2720) may be bent within articulation section (1736), manually by a user before insertion, or with a conventional grasper while end effector (2740) is positioned within a patient. Other suitable structures and techniques that may be used to contain bearings (2730) and wire (2760) through an articulation section will be apparent to those of ordinary skill in the art in view of the teachings herein.

In an exemplary use, end effector (2740) is inserted into a patient via a trocar. Articulation section (2736) is substantially straight when end effector (2740) and part of shaft (2720) are inserted through the trocar, such that end effector (2740) is substantially aligned with shaft (2720). As described above, articulation cables (1710, 1712) may be pushed and/or pulled to pivot articulation section (1736) and bend shaft (2720). Or a grasper may be used to bend shaft (2720) to a desired position and orientation relative to an anatomical structure within the patient. Two layers of tissue of the anatomical structure are then captured between jaws (2742, 2744) by squeezing trigger (24) toward pistol grip (22). Squeezing trigger (24) pushes jaw cable (2748). As jaw cable (2748) translates distally, ramp surface (2745) of upper jaw (2742) slides along sphere (2746) of jaw cable (2748) to substantially close jaws (2742, 2744) by pivoting jaw (2742) relative to jaw (2744), as shown in FIG. 11B. Squeezing trigger (24) also translates bearings (2730) distally to translate blade member (2768) distally. Actuation of jaw cable (2748) and bearings (2730) may be staged (e.g., trigger (24) may be squeezed through a first range of motion to actuate jaw cable (2748) to close jaws (2742, 2744), then trigger (24) may be squeezed through a second range of motion to actuate bearings (2730) to drive blade (2764)). Alternatively, separate actuators may be provided on handpiece (20) to independently actuate jaw cable (2748) and bearings (2730). Flanges (2762, 2766) cammingly act to close jaws (2742, 2744) further.

With tissue layers captured between jaws (2742, 2744), bearings (2730) continue to advance distally by the user squeezing trigger (24) toward pistol grip (22), as shown in FIG. 11C. As bearings (2730) advance distally, distal blade (2764) simultaneously severs the clamped tissue layers, resulting in separated upper layer portions being apposed with respective separated lower layer portions. Flanges (2762, 2766) help maintain a significantly compressive force between jaws (2742, 2744). With severed tissue layer portions being compressed between jaws (2742, 2744), electrode surfaces (50, 52) are activated with bipolar RF energy by the user depressing activation button (26). Bipolar RF energy delivered by power source (80) ultimately thermally welds the tissue layer portions on one side of blade member (2768) together and the tissue layer portions on the other side of blade member (2768) together. After the tissue layer portions have been welded, wire (2760) may be translated proximally to retract blade member (2768) and bearings (2730). Jaw cable (2748) may be translated proximally to pivot jaw (2742) relative to jaw (2744) to open jaws (2742, 2744).

C. Exemplary Looped Cable Cutting Member Actuation

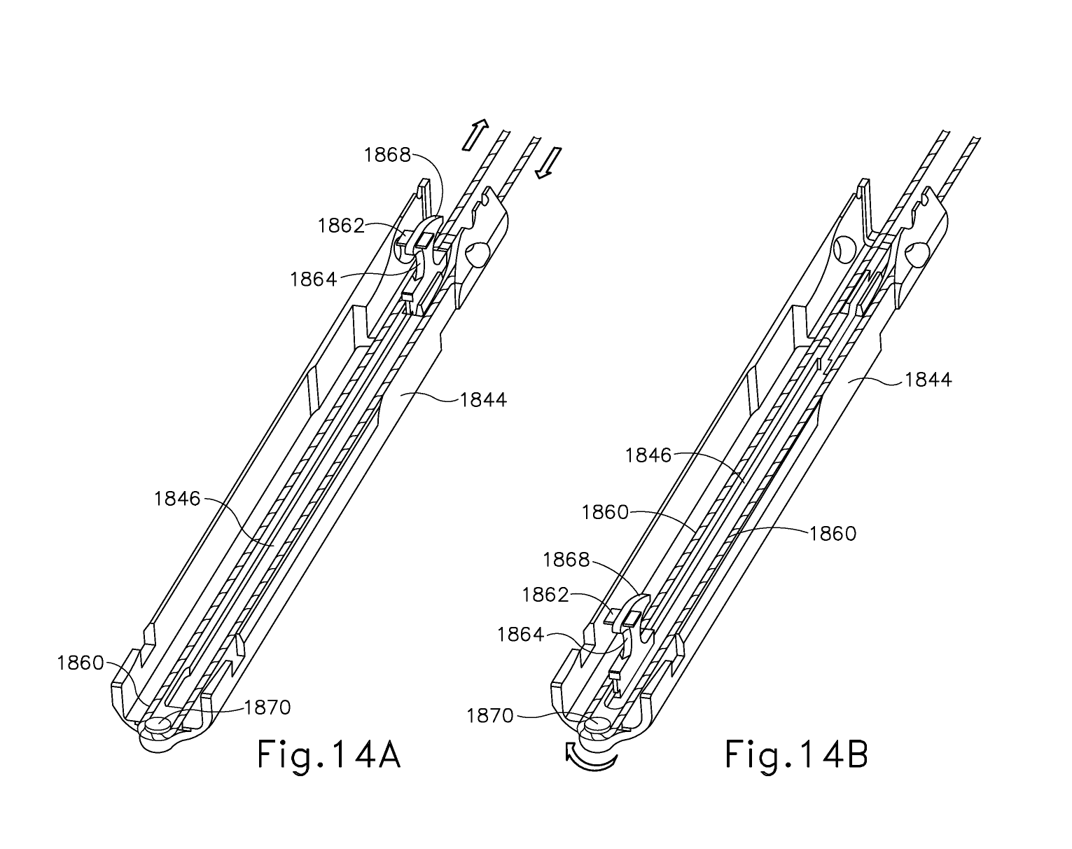

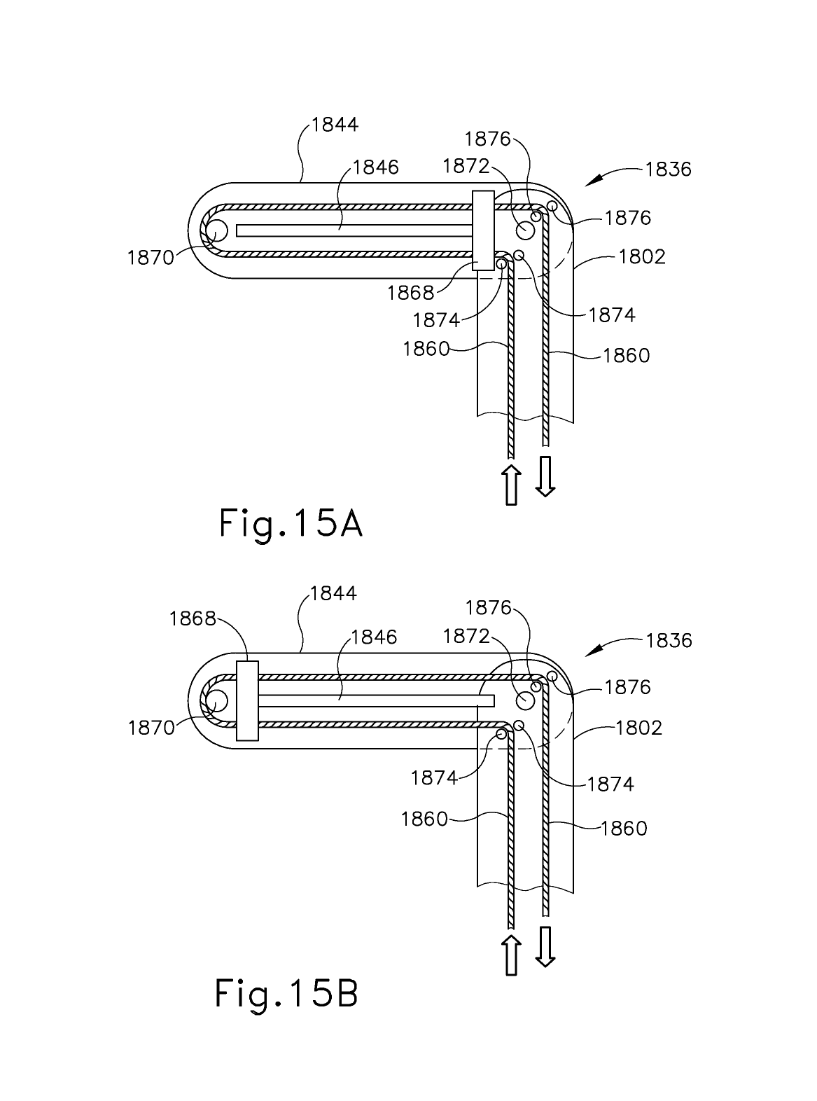

Alternatively, a cable (1860) looped through lower jaw (1844) is used to translate blade member (1868) as shown in FIGS. 14A-15B. Lower jaw (1844) is similar to lower jaw (1644) in FIGS. 6A-6B. In this example, blade (1864) is distally presented by a blade member (1868). Blade member (1868) travels longitudinally along a slot (1846) formed in a lower jaw (1844) of an end effector assembly. Blade member (1868) further includes lower flange members (not shown) that are disposed beneath lower jaw (1844). By way of example only, lower jaw (44) of electrosurgical instrument (10) may be readily modified to include the features of lower jaw (1844) described in this example.

The distal end of lower jaw (1844) includes a post (1870). Cable (1860) is wrapped around post (1870) such that post (1870) redirects the longitudinal motion of cable (1860) by approximately 180°. Cable (1860) has sufficient flexibility to provide such motion, yet also has enough tensile strength to bear significant loads on blade member (1868) as blade (1864) severs tissue. In some versions, post (1870) includes a bushing or bearing that is configured to facilitate movement of cable (1860) about post (1870). As can be seen from the transition between FIG. 14A and FIG. 14B, blade member (1868) advances distally along channel (1846) from a proximal position to a distal position in response to movement of cable (1860). For instance, one side of blade member (1868) is secured to cable (1860) such that pulling one end of cable (1860) proximally and/or pushing the other end of cable (1860) distally translates blade member (1868). In some versions, the ends of cable (1860) may also be translated in the other direction to return blade member (1868) from the distal position to the proximal position.

An exemplary articulation section (1836) is shown in FIGS. 15A-15B. Lower jaw (1844) is articulated relative to shaft (1802) via pivoting coupling (1872). Cable (1860) extends through shaft (1802), around post (1870) of lower jaw (1844), and back through shaft (1802). Lower jaw (1844) also comprises a pair of guide posts (1874) at the proximal end on a first side, and a pair of guide posts (1876) at the proximal end on a second, opposing side. Cable (1860) extends through each pair of guide posts (1874, 1876) to prevent cable (1860) from binding as articulation section (1836) is articulated and as cable (1860) is translated.

D. Exemplary Rotary Cutting Member Actuation

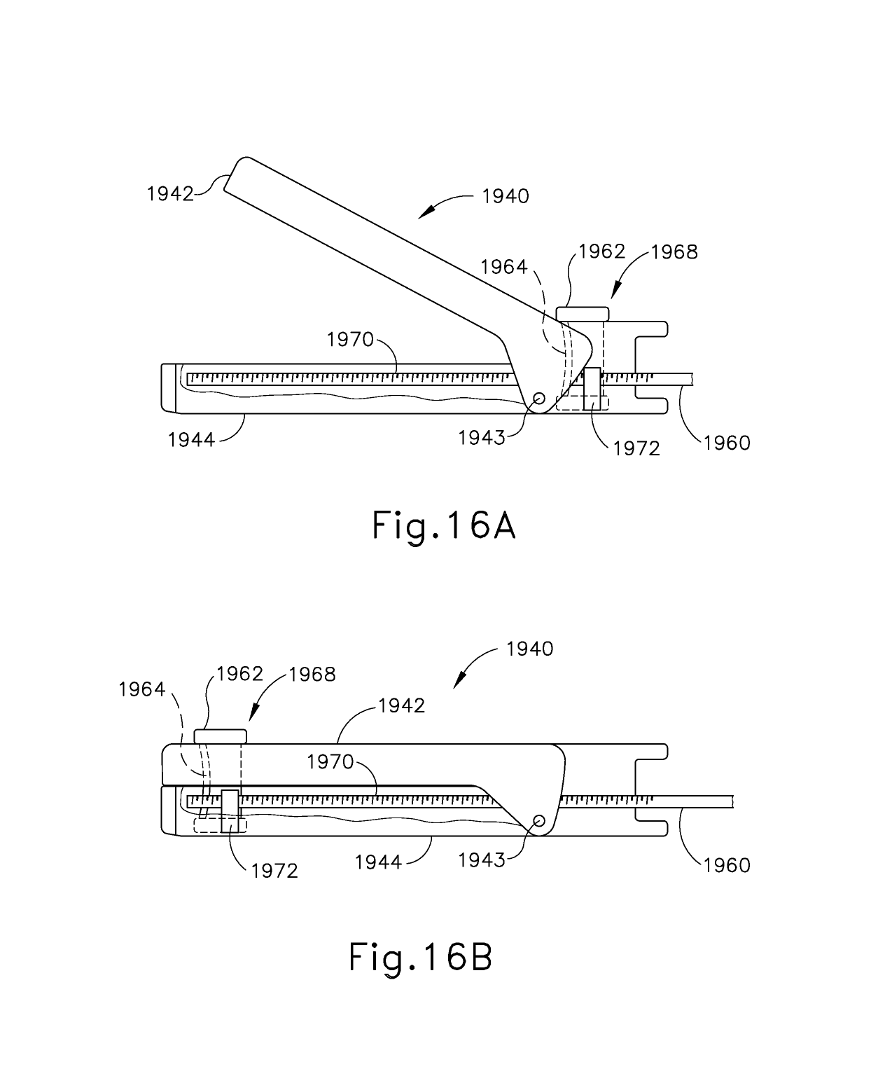

Alternatively, a rotary drive is used to translate blade member (1968) as shown in FIGS. 16A-16B. End effector (1940) of this example is similar to end effector (40), except that lower jaw (1944) of end effector (1940) comprises a screw drive (1970). Screw drive (1970) extends through lower jaw (1944) and is configured to rotate within lower jaw (1944). Shaft (1960) extends proximally from screw drive (1970). Shaft (1960) may be rotated by a rotary motor (e.g. from handpiece (20), etc.) or manually. Such rotation is communicated to screw drive (1970). Shaft (1960) is flexible and configured to bend through a significantly articulated articulation section (36). Blade member (1968) is coupled to screw drive (1970). Blade member (1968) is similar to blade member (1768), except that blade member (1968) comprises a nut (1972) at the lower end of blade member (1968). Nut (1972) comprises threads that correspond to screw (1970) and is configured to wrap around screw drive (1970). Accordingly, when screw drive (1970) is rotated, nut (1972) translates blade member (1978). Because nut (1972) translates through lower jaw (1944) and upper flange (1962) is positioned on a top surface of upper jaw (1942), nut (1972) and flange (1962) cammingly act to pivot jaw (1942) toward jaw (1944) when blade member (1968) is translated distally, as shown in FIG. 16B.

In an exemplary use, end effector (1940) is inserted into a patient via a trocar. Articulation section (36) is substantially straight when end effector (1940) and part of shaft (30) are inserted through the trocar, such that end effector (1940) is aligned with shaft (30). Articulation control (28) may then be manipulated to pivot or flex articulation section (36) of shaft (30) in order to position end effector (1940) at a desired position and orientation relative to an anatomical structure within the patient. Two layers of tissue of the anatomical structure are then captured between jaws (1942, 1944) by squeezing trigger (24) toward pistol grip (22). Squeezing trigger (24) causes shaft (1960) to rotate, thereby rotating screw drive (1970). Rotation of screw drive (1970) advances nut (1972) distally along screw drive (1970) to translate blade member (1968) distally. Nut (1972) and flange (1962) cammingly act to close jaws (1942, 1944), as shown in FIG. 16B.

As blade member (1968) advances distally, distal blade (1964) simultaneously severs the clamped tissue layers, resulting in separated upper layer portions being apposed with respective separated lower layer portions. With severed tissue layer portions being compressed between jaws (1942, 1944), electrode surfaces (50, 52) are activated with bipolar RF energy by the user depressing activation button (26). Bipolar RF energy delivered by power source (80) ultimately thermally welds the tissue layer portions on one side of blade member (1968) together and the tissue layer portions on the other side of blade member (1968) together. After the tissue layer portions have been welded, shaft (1960) may be rotated in the other direction, thereby rotating screw drive (1970) in the other direction. Screw drive (1970) then translates nut (1970) proximally to retract blade member (1968).

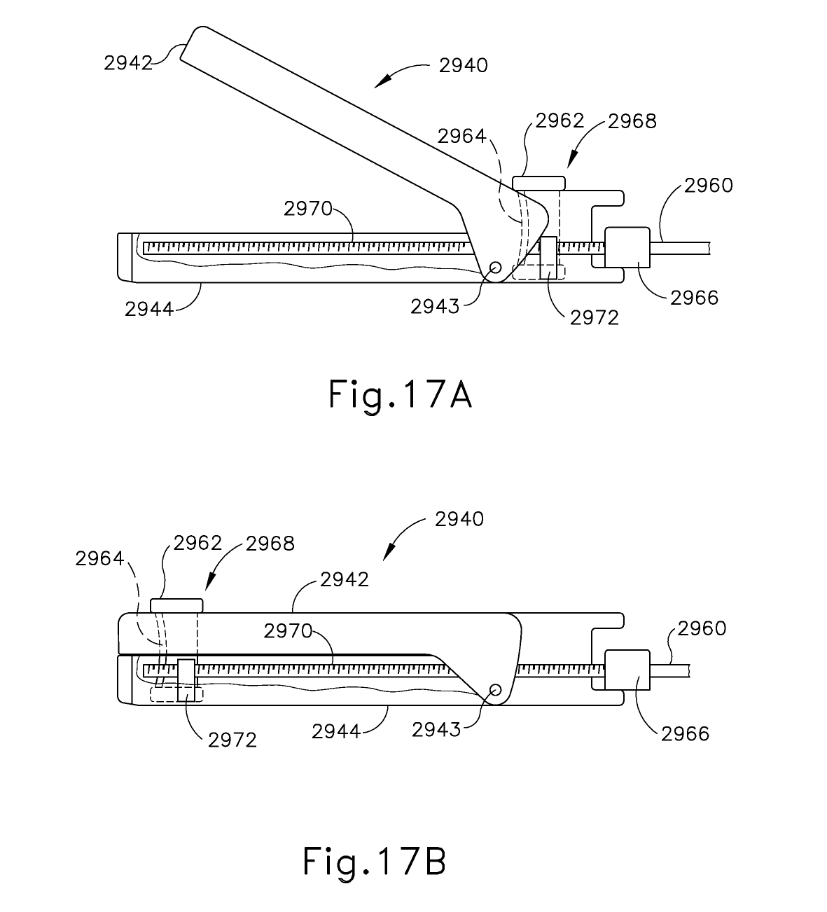

Alternatively, a rigid shaft (2960) may be used to rotate a screw drive (2970), as shown in FIGS. 17A-17B. End effector (2940) of this example is similar to end effector (1940), except that end effector (2940) comprises a rigid shaft (2960) and a gear assembly (2966). Screw drive (2970) extends through lower jaw (2944) and is configured to rotate. Shaft (2960) is positioned proximally to screw drive (2970) and is operably linked to screw drive (2970) such that rotation of shaft (2960) rotates screw drive (2970). Gear assembly (2966) couples shaft (2960) to screw drive (2970) such that rotation of shaft (2960) is communicated through a significantly articulated articulation section (36). In some versions, gear assembly (2966) comprises one or more universal joints and/or similar types of joints or couplings. By way of example only, gear assembly (2966) may comprise internal threading engaged with screw drive (2970). In addition or in the alternative, gear assembly (2966) may comprise features pressed against each other in an end-to-end configuration, such that deformation and/or friction between the pressed features provides transmission of rotation from one of the features to the other of the features. As yet another merely illustrative example, gear assembly (2966) may comprise meshing face gears at the ends of shaft (2960) and screw drive (2970). Other suitable components and configurations that may be used for gear assembly (2966) will be apparent to those of ordinary skill in the art in view of the teachings herein.

Shaft (2960) may be rotated by a rotary motor (e.g. from handpiece (20), etc.) or manually. Blade member (2968) is coupled to screw drive (2970) by nut (2972). Nut (2972) comprises threads that correspond to screw drive (2970) and is configured to wrap around screw drive (2970). Accordingly, when screw drive (2970) is rotated by shaft (2960) via gear assembly (2966), nut (2972) translates blade member (2978). Because nut (2972) translates through lower jaw (2944) and upper flange (2962) is positioned on a top surface of upper jaw (2942), nut (2972) and flange (2962) cammingly act to pivot jaw (2942) toward jaw (2944) when blade member (2968) is translated distally, as shown in FIG. 17B. Shaft (2960) may be rotated in the opposite direction to translate blade member (2968) proximally. For example, shaft (2960) may be rotated clockwise to advance blade member (2968) distally and shaft (2960) may be rotated counterclockwise to retract blade member (2968) proximally. Suitable rotation configurations will be apparent to one with ordinary skill in the art in view of the teachings herein.

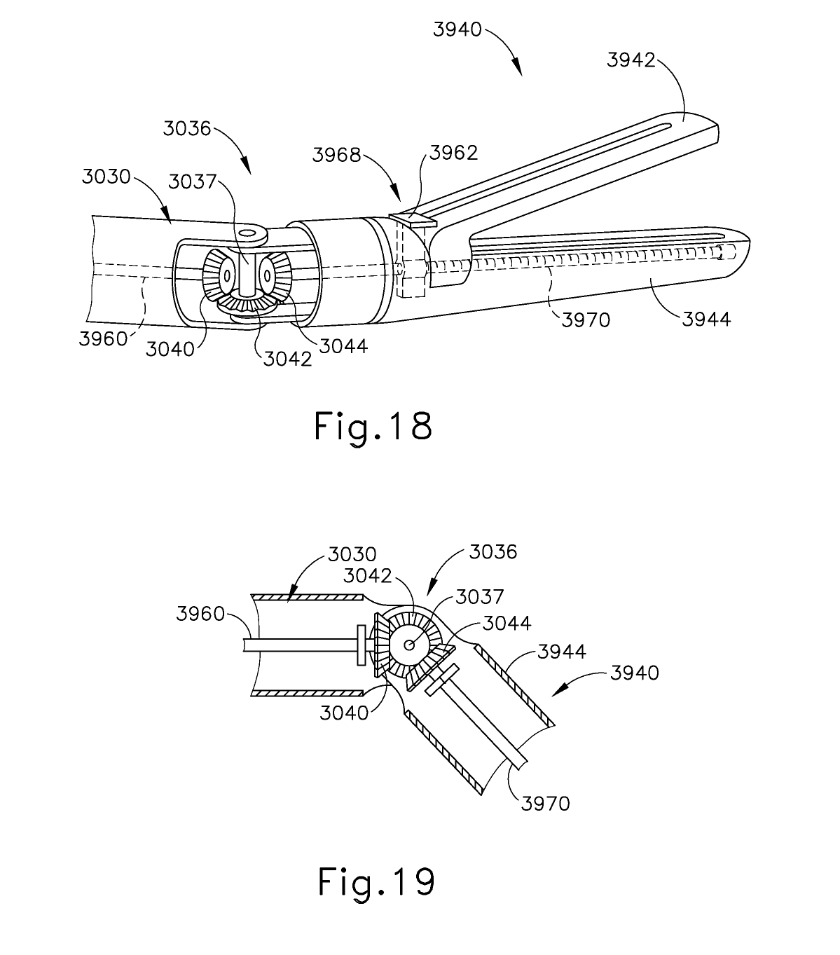

FIGS. 18-19 show another exemplary end effector (3940), which is substantially similar to end effector (2940) described above. End effector (3940) of this example comprises an upper jaw (3942), a lower jaw (3944), and a blade member (3968). Blade member (3968) includes an upper flange (3962) that bears down against upper jaw (3942) as blade member (3968) is translated distally within jaws (3942, 3944), to thereby pivot upper jaw (3942) downwardly toward lower jaw (3944). Blade member (3968) is threadably coupled with a screw drive (3970), such that blade member (3968) translates longitudinally in response to rotation of screw drive (3970).

End effector (3940) is coupled to a shaft assembly (3030) by an articulation joint (3036). Articulation joint (3036) enable end effector (3940) to be selectively deflected away from the longitudinal axis of shaft assembly (3030). Articulation joint (3036) of the present example comprises a vertical pin (3037) passing through complementary clevis features. It should be understood that articulation joint (3036) may be configured and/or driven in accordance with any of the teachings herein, in accordance with any of the teachings of any of the various references that are incorporated by reference herein, and/or in any other suitable fashion. A rigid shaft (3960) extends through shaft assembly (3030) and terminates at a distally facing bevel gear (3040), such that bevel gear (3040) rotates when shaft (3960) rotates. Bevel gear (3040) meshes with another bevel gear (3042), which rotates about the axis defined by pin (3037). Yet another bevel gear (3044) meshes with bevel gear (3042). Bevel gear (3044) is secured to the proximal end of screw drive (3970), such that bevel gear (3044) and screw drive (3970) rotate together. Bevel gears (3040, 3042, 3044) all rotate together due to their meshing relationships with each other. It should therefore be understood that rotation of shaft (3960) will be transmitted to screw drive (3970) via bevel gears (3040, 3042, 3044). Bevel gears (3040, 3042, 3044) may thus be together viewed as a variation of gear assembly (2966) described above. It should also be understood that the configuration of bevel gears (3040, 3042, 3044) may enable transmission of rotation from shaft (3960) to screw drive (3970) regardless of the angle at which end effector (3940) is articulated relative to shaft assembly (3030). Still other suitable components, features, configurations, and arrangements will be apparent to those of ordinary skill in the art in view of the teachings herein.

Various examples described herein include components that extend through an articulation section to an end effector and that may be formed of electrically conductive materials, including but not limited to various firing beams, firing bands, support beams, articulation beams, articulation cables, etc. Any such components may be used to provide electrical communication to a component of an end effector. By way of example only such components may be used to communicate power to the end effector from a power source, to provide a ground return path, to communicate signals to or from the end effector, etc. Of course, such components may also include appropriate insulation as needed or desired. Various suitable ways in which such components may be used to communicate power, signals, etc. through an articulation section will be apparent to those of ordinary skill in the art in view of the teachings herein.

It should be understood that any of the devices herein may also include one or more of the various features disclosed in U.S. Pub. No. 2012/0078243, entitled “Control Features for Articulating Surgical Device,” published Mar. 29, 2012, the disclosure of which is incorporated by reference herein; U.S. Pub. No. 2012/0078244, entitled “Control Features for Articulating Surgical Device,” published Mar. 29, 2012, the disclosure of which is incorporated by reference herein; U.S. Pub. No. 2012/0078247, entitled “Articulation Joint Features for Articulating Surgical Device,” published Mar. 29, 2012, the disclosure of which is incorporated by reference herein; U.S. Pub. No. 2012/0078248, entitled “Articulation Joint Features for Articulating Surgical Device,” published Mar. 29, 2012, the disclosure of which is incorporated by reference herein; and/or U.S. patent application Ser. No. 13/622,735, entitled “Surgical Instrument with Contained Dual Helix Actuator Assembly,” filed on Sep. 19, 2012, the disclosure of which is incorporated by reference herein.

It should also be understood that any of the devices described herein may be modified to include a motor or other electrically powered device to drive an otherwise manually moved component. Various examples of such modifications are described in U.S. Pub. No. 2012/0116379, entitled “Motor Driven Electrosurgical Device with Mechanical and Electrical Feedback,” published on May 10, 2012, the disclosure of which is incorporated by reference herein. Various other suitable ways in which a motor or other electrically powered device may be incorporated into any of the devices herein will be apparent to those of ordinary skill in the art in view of the teachings herein.

Furthermore, it should be understood that any of the devices described herein may be modified to contain most, if not all, of the required components within the medical device itself. More specifically, the devices described herein may be adapted to use an internal or attachable power source instead of requiring the device to be plugged into an external power source by a cable. Various examples of how medical devices may be adapted to include a portable power source are disclosed in U.S. Provisional Application Ser. No. 61/410,603, filed Nov. 5, 2010, entitled “Energy-Based Surgical Instruments,” the disclosure of which is incorporated by reference herein. Various other suitable ways in which a power source may be incorporated into any of the devices herein will be apparent to those of ordinary skill in the art in view of the teachings herein.

V. Miscellaneous

While the examples herein are described mainly in the context of electrosurgical instruments, it should be understood that the teachings herein may be readily applied to a variety of other types of medical instruments. By way of example only, the teachings herein may be readily applied to tissue graspers, tissue retrieval pouch deploying instruments, surgical staplers, ultrasonic surgical instruments, etc. It should also be understood that the teachings herein may be readily applied to any of the instruments described in any of the references cited herein, such that the teachings herein may be readily combined with the teachings of any of the references cited herein in numerous ways. Other types of instruments into which the teachings herein may be incorporated will be apparent to those of ordinary skill in the art.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Embodiments of the present invention have application in conventional endoscopic and open surgical instrumentation as well as application in robotic-assisted surgery. For instance, those of ordinary skill in the art will recognize that various teaching herein may be readily combined with various teachings of U.S. Pat. No. 6,783,524, entitled “Robotic Surgical Tool with Ultrasound Cauterizing and Cutting Instrument,” published Aug. 31, 2004, the disclosure of which is incorporated by reference herein.

Embodiments of the devices disclosed herein can be designed to be disposed of after a single use, or they can be designed to be used multiple times. Embodiments may, in either or both cases, be reconditioned for reuse after at least one use. Reconditioning may include any combination of the steps of disassembly of the device, followed by cleaning or replacement of particular pieces, and subsequent reassembly. In particular, embodiments of the device may be disassembled, and any number of the particular pieces or parts of the device may be selectively replaced or removed in any combination. Upon cleaning and/or replacement of particular parts, embodiments of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, embodiments described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a medical facility. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus for operating on tissue, the apparatus comprising:
(a) an end effector, the end effector comprising:
(i) a first jaw,
(ii) a second jaw, wherein the first jaw is configured to pivot relative to the second jaw from an open position and a closed position, and
(iii) a blade member, wherein the blade member is configured to translate within the end effector;
(b) a shaft, wherein the shaft defines a longitudinal axis;
(c) an articulation portion positioned between the end effector and the shaft, wherein the articulation portion is configured to deflect the end effector away from the longitudinal axis of the shaft; and
(d) a translating cable extending through the articulation portion, wherein the translating cable is coupled with the blade member, wherein the translating cable is operable to translate the blade member longitudinally relative to the first and second jaws.

2. The apparatus of claim 1, further comprising a set of ball members associated with the translating cable, wherein the ball members are configured to translate with the translating cable.

3. The apparatus of claim 2, wherein the translating cable extends through the ball members.

4. The apparatus of claim 2, wherein the ball members are operable to drive the blade member distally.

5. The apparatus of claim 2, wherein the translating cable is operable to pull the blade member proximally.

6. The apparatus of claim 2, wherein the articulation portion comprises a pre-bent tube.

7. The apparatus of claim 6, wherein the ball members are positioned in the pre-bent tube.

8. The apparatus of claim 2, wherein the second jaw defines a channel configured to receive at least some of the ball members.

9. The apparatus of claim 1, one of the first jaw or the second jaw includes a guide post, wherein the translating cable is at least partially wrapped about the guide post.

10. The apparatus of claim 9, wherein the one of the first jaw or the second jaw has a distal end, wherein the guide post is positioned at the distal end of the one of the first jaw or the second jaw.

11. The apparatus of claim 9, wherein the guide post is operable to redirect the cable by approximately 180 degrees.

12. The apparatus of claim 1, wherein the translating cable is operable to advance the blade member distally in response to proximal movement of the translating cable.

13. The apparatus of claim 1, wherein a distal end of the translating cable is secured to the blade member.

14. The apparatus of claim 1, further comprising a jaw opening cable, wherein the jaw opening cable is translatable to pivot the first jaw relative to the second jaw.

15. The apparatus of claim 14, wherein the jaw opening cable comprises a ball end coupled with the first jaw.

16. The apparatus of claim 1, wherein the translating cable defines a first length and a second length extending through the articulation portion, wherein the first and second lengths are configured to translate in opposing directions in response to translation of the blade member.

17. The apparatus of claim 1, wherein the articulation portion is configured to bend more than about 90 degrees.

19. An apparatus for operating on tissue, the apparatus comprising:
(a) an end effector, the end effector comprising:
(i) a first jaw,
(ii) a second jaw, wherein the first jaw is configured to pivot relative to the second jaw from an open position and a closed position, wherein at least one of the first jaw or the second jaw comprises an electrode, and
(iii) a blade member, wherein the blade member is configured to translate within the end effector;
(b) a shaft, wherein the shaft defines a longitudinal axis; and
(c) an articulation portion positioned between the end effector and the shaft, wherein the articulation portion is configured to deflect the end effector away from the longitudinal axis of the shaft, wherein the articulation portion comprises an actuator operable to translate the blade member, wherein the actuator comprises one or both of a cable or bearings.

20. An apparatus for operating on tissue, the apparatus comprising:
(a) an end effector, the end effector comprising:
(i) a first jaw,
(ii) a second jaw, wherein the first jaw is configured to pivot relative to the second jaw from an open position and a closed position, wherein the second jaw comprises a screw drive, and
(iii) a blade member, wherein the blade member is configured to translate within the end effector, wherein the blade member comprises a threaded feature coupled with the screw drive;
wherein at least one of the jaws comprises at least one electrode, wherein the at least one electrode is operable to deliver RF energy to tissue clamped between the first and second jaw;
(b) a shaft; and
(c) an articulation portion positioned between the end effector and the shaft, wherein the articulation portion is configured to deflect the end effector away from the longitudinal axis of the shaft, wherein the articulation portion comprises a drive shaft coupled with the screw drive, wherein the drive shaft is operable to rotate the screw drive to translate the threaded feature of the blade member along the screw drive.
